(19) United States
(12) Patent Application Publication
(10) Pub. No.: US 2024/0066091 A1
(43) Pub. Date: Feb. 29, 2024

(54) USE OF PLANT-DERIVED EXOSOMES FOR INDUCING DIFFERENTIATION OF STEM CELL SOURCES INTO CARTILAGE AND BONE CELLS

(71) Applicant: YEDITEPE UNIVERSITESI, Istanbul (TR)

(72) Inventors: Fikrettin SAHIN, Istanbul (TR); Polen KOCAK, Istanbul (TR); Merve YILDIRIM, Istanbul (TR)

(73) Assignee: YEDITEPE UNIVERSITESI, Istanbul (TR)

(21) Appl. No.: 18/270,844

(22) PCT Filed: Dec. 27, 2021
(86) PCT No.: PCT/TR2021/051534

§ 371 Date: Jul. 4, 2023

(30) Foreign Application Priority Data
Jan. 4, 2021 (TR) 2021/00031

(51) Int. Cl. A61K 36/899; A61K 9/127; A61K 35/28; A61P 19/04; C12N 5/077

(52) U.S. Cl. CPC A61K 36/899; A61K 9/127; A61K 35/28; A61P 19/04; C12N 5/0654; C12N 5/0655; C12N 2500/76; C12N 2501/734; C12N 2506/1384

(57) ABSTRACT

A product including plant-derived nanovesicles which induce differentiation of the stem cell sources into cartilage and bone cells is provided. The objective of the invention is to improve the rate of differentiation into cartilage and bone cells, enhance strength of the obtained tissue and to provide a non-toxic application with lower cost by means of giving plant exosomes in addition to the differentiation media to the adipose-derived stem cells.

### CROSS REFERENCE TO THE RELATED APPLICATIONS

This application is the national phase entry of International Application No. PCT/TR2021/051534, filed on Dec. 27, 2021, which is based upon and claims priority to Turkish Patent Application No. 2021/00031, filed on Jan. 4, 2021, the entire contents of which are incorporated herein by reference.

### TECHNICAL FIELD

The present invention relates to a product comprising plant-derived nanovesicles which induce differentiation of the stem cell sources into cartilage and bone cells.

### BACKGROUND

Cartilage is a resilient and smooth elastic tissue, which protects the ends of long bones at the joints, and serves as a structural component of the rib cage, the ear, the nose, the bronchial tubes, the intervertebral discs, and many other body components. The matrix of cartilage is made up of glycosaminoglycans, proteoglycans, collagen fibers and sometimes elastin. Cartilage does not contain blood vessels or nerves. When compared to the other connective tissues, it has a very slow turnover of its extracellular matrix and is hardly repaired.

Bone and cartilage damage are treatments that take time to heal. Being bedridden due to fractures, especially at advanced ages, generally results in patient deaths. There is no effective product developed in this regard. Likewise, there are not many effective products for diseases that are difficult to treat such as osteoporosis and osteoarthritis. There is no supportive product for osteoporosis, which is one of the side effects of chemotherapy.

Vesicles are sac-shaped structures with a closed and bilayered lipid membrane which enable the storage and/or transport of substances. While vesicles can be formed naturally within the cell structure, they can also be prepared artificially to achieve these purposes. Vesicles usually have a certain size and a standard structure.

Exosomes are extracellular vesicles (EVs) of 40-120 nm size having a protein and lipid membrane. EVs are frequently produced within the endosomal compartment of eukaryotic cells. In multicellular organisms, exosomes and other extracellular vesicles are found within tissues and can also be found in biological fluids such as blood, urine, and spinal fluid. They are responsible for intercellular messaging. They can also be released into a culture conditioned medium in vitro by cultured cells. Today's studies have proven that parasites, microorganisms and plants also secrete exosomes.

The European patent document numbered EP3235500, an application in the state of the art, discloses a composition for inducing chondrocyte differentiation or regenerating cartilage tissue, including exosomes extracted from stem cells differentiating into chondrocytes. It can be used as a medium composition for inducing chondrocyte differentiation, an injection preparation for regenerating cartilage tissue, and a pharmaceutical composition for treating cartilage disorders. Stem cells that will differentiate into chondrocytes may be the bone marrow stem cells, the umbilical cord blood stem cells, and the adipose-derived stem cells and these stem cells may be stem cells derived from a human, an animal, or a plant.

South Korean patent application document no. KR20130079673, an application in the state of the art, discloses a composition for improving stem cell differentiation. This composition contains an Epimedii herba extract and improves stem cell differentiation potency.

South Korean patent application document no. KR20180092348, an application in the state of the art, discloses a composition for improving differentiation of chondrocytes and regenerating cartilage tissues, which includes stem cells derived from cord blood or umbilical cords that differentiate into the chondrocytes.

### SUMMARY

The objective of the invention is to improve differentiation into cartilage and bone cells and to shorten the differentiation time by giving plant exosomes as well as differentiation media to adipose-derived stem cells.

Another objective of the invention is to develop a product which is low cost and nontoxic thanks to the fact that it is plant-derived.

### BRIEF DESCRIPTION OF THE DRAWINGS

“Use of plant exosomes for inducing differentiation of stem cell sources into cartilage and bone cells” developed to fulfill the objectives of the present invention is illustrated in the accompanying figures, in which:

FIG. 1 is an image of the wheat-derived exosome under a scanning electron microscope.

FIGS. 2A-2E are flow cytometric characterizations of wheat-derived exosome with HSP 70, CD 9, CD 81 and CD 63 surface markers.

FIG. 2A is a flow cytometric characterization of wheat-derived exosome.

FIG. 2B is a flow cytometric characterization of wheat-derived exosome with CD 9 surface markers.

FIG. 2C is a flow cytometric characterization of wheat-derived exosome with CD 63 surface marker.

FIG. 2D is a flow cytometric characterization of wheat-derived exosome with CD81 surface marker.

FIG. 2E is a flow cytometric characterization of wheat-derived exosome with HSP 70 surface marker.

FIG. 3A is a flow cytometric characterization of human-derived adipose cells (hASCs) with control (NC) surface marker.

FIG. 3B is a flow cytometric characterization of human-derived adipose cells (hASCs) with CD90 surface marker.

FIG. 3C is a flow cytometric characterization of human-derived adipose cells (hASCs) with CD73 surface marker.

FIG. 3D is a flow cytometric characterization of human-derived adipose cells (hASCs) with CD44 surface marker.

FIG. 3E is a flow cytometric characterization of human-derived adipose cells (hASCs) with CD105 surface marker.

FIG. 3F is a flow cytometric characterization of human-derived adipose cells (hASCs) with Integrin 131 surface marker.

FIG. 3G is a flow cytometric characterization of human-derived adipose cells (hASCs) with CD29 surface marker.

FIG. 3H is a flow cytometric characterization of human-derived adipose cells (hASCs) with CD34 surface marker.

FIG. 3I is a flow cytometric characterization of human-derived adipose cells (hASCs) with CD45 surface marker.

FIG. 3J is a flow cytometric characterization of human-derived adipose cells (hASCs) with CD10 surface marker.

FIG. 3K is a flow cytometric characterization of human-derived adipose cells (hASCs) with CD31 surface marker.

FIG. 4A is an image of differentiation of control group human-derived adipose cell (hASCs) into bone cell stained with Alizarin Red under light microscope.

FIG. 4B is an image of differentiation of human-derived adipose cell (hASCs), on which 100 μg/ml wheat-derived exosome is applied, into bone cell stained with Alizarin Red under light microscope.

FIG. 4C is an image of differentiation of human-derived adipose cell (hASCs), on which 200 μg/ml wheat-derived exosome is applied, into bone cell stained with Alizarin Red under light microscope.

FIG. 5A Image of differentiation of human-derived adipose cell (hASCs), on which 200 μg/ml wheat-derived exosome is applied, into bone cell stained with Alizarin Red under light microscope.

FIG. 5B Image of differentiation of human-derived adipose cell (hASCs), on which 200 μg/ml wheat-derived exosome is applied, into bone cell stained with Alizarin Red under light microscope.

FIG. 5C Image of differentiation of human-derived adipose cell (hASCs), on which 200 μg/ml wheat-derived exosome is applied, into bone cell stained with Alizarin Red under light microscope.

FIG. 6A is an image of the varying gene expressions of the cells differentiating from human-derived adipose cells (hASCs) into bone, according to the result of real-time PCR performed with the Osteocalcin (OCN) gene.

FIG. 6B is an image of the varying gene expressions of the cells differentiating from human-derived adipose cells (hASCs) into bone, according to the result of real-time PCR performed with the Collagen1-A (COL1A) gene.

FIG. 6C is an image of the varying gene expressions of the cells differentiating from human-derived adipose cells (hASCs) into bone, according to the result of real-time PCR performed with the Alkaline Phosphatase (ALP) gene.

FIG. 7A is an image of the varying gene expressions of the cells differentiating from human-derived adipose cells (hASCs) into bone, according to the result of real-time PCR performed with the Collagen 2-A (COL1A) gene.

FIG. 7B is an image of the varying gene expressions of the cells differentiating from human-derived adipose cells (hASCs) into bone, according to the result of real-time PCR performed with the Campomelic Dysplasia (SOX9) gene.

### DETAILED DESCRIPTION OF THE EMBODIMENTS

Within the scope of the invention, in the differentiation of hASCs (adipose derived), tooth root, bone marrow, cartilage, endometrium, cord blood and IPS (induced pluripotent stem cells) stem cells into bone and cartilage cells at the same time, plant exosomes are given to the stem cells in addition to the differentiation media, thereby accelerating differentiation into cartilage and bone cells and strengthening the formed bone and cartilage. In order to ensure differentiation in the hASCs (Adipose-derived stem cell) cell line into cartilage and bone cells, plant exosomes (at concentrations of 100 μg/ml and 200 μg/ml) are given to the cells that are seeded in 6-well culture plates in addition to the differentiation media. This procedure is repeated by changing the media of all cells every 3 days, and at the end of 15 days, the wells are first washed with PBS and then incubated in 4% Paraformaldehyde for 20 minutes whereby the cells are fixed on the surface.

In one embodiment of the invention, plant-derived exosomes are used to accelerate the recovery after osteoarthritis, osteoporosis, bone fracture, cartilage destruction, bone and cartilage surgeries up to 3-4 weeks, as they accelerate the differentiation of adipose stem cells into bone and/or cartilage tissue.

Within the scope of the invention, a product comprising the said plant-derived exosomes is in the form of serum, syrup, tablet, drug, gel, and cream.

Within the scope of the invention, plant-derived exosomes are obtained by means of the following process steps:
Grinding the plant-derived sample in 1% PBS (phosphate buffered saline),
Then filtering,
Centrifugation of the obtained filtrate,
Isolating the exosomes by using exosome isolation kit or aqueous two-phase system (ATPS) method,
Dissolving the isolated exosomes in 0.9% isotonic serum.

Within the scope of the invention, wheatgrass is used as the plant source for obtaining the plant-derived exosomes. During the preliminary sample preparation, it was determined that it was adequate for the seeds to grow for a period of 1.5 weeks.

On the other hand, human-derived adipose stem cells, which are also included in the scope of the invention, are obtained by means of the following process steps:
Cutting the adipose tissues that are surgically removed from a human into small pieces by the help of a bistoury,
Adding 5 mg/ml of Collagenase type II enzyme into 10 mL of PBS and incubating at 37° C. for 3 hours,
Centrifugation of the cells at 400×g after incubation,
Seeding the cells and tissues into cell culture plates with antibiotic-containing media and ensuring adhesion of the cells for 3-4 days.

### Experimental Studies

### Preparation of Plant-Derived Exosome

In the inventive product containing plant-derived exosome, wheatgrass is used as the plant source. The wheatgrass preferably selected in the experimental studies was obtained from Turkey, Adana Ceyhan 69 seeds. During the preliminary sample preparation, it was determined that it was adequate for the seeds to grow for a period of 1.5 weeks.

The collected wheatgrass was first ground in 1% PBS (phosphate buffered saline) and then filtered. The obtained filtrate wheatgrass was centrifuged at 1000×g for 10 minutes, 3200×g for 20 minutes, 15000×g for 60 minutes, and then was isolated by using Exospin exosome isolation kit. The isolated exosomes were dissolved in 0.9% isotonic serum.

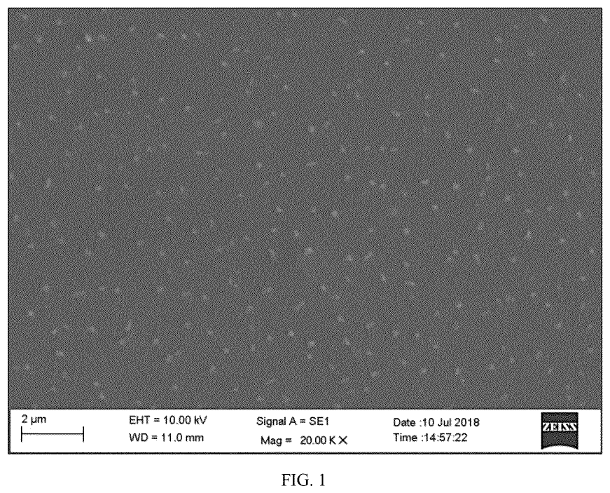

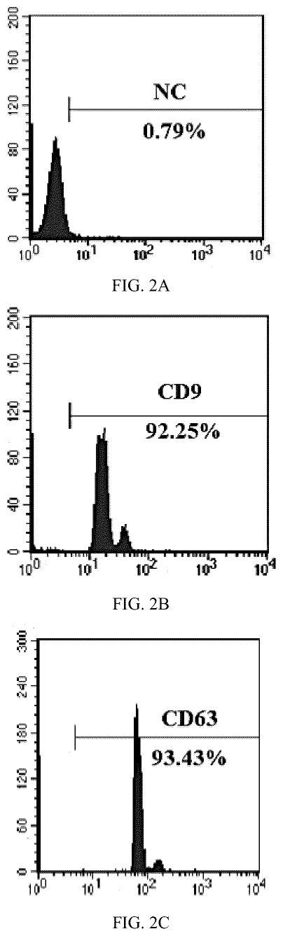

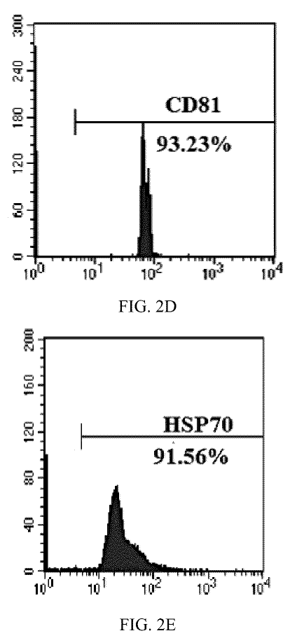

The inventive product exosomes, which were isolated and were in the serum form, were observed by using scanning electron microscope (FIG. 1). Then they were incubated with HSP 70, CD 9, CD 81 and CD 63 surface markers and thereby flow cytometric characterization of the exosomes was performed. (FIGS. 2A-2E)

### Obtaining Human-Derived Adipose Stem Cells

The adipose tissues surgically removed from a human were cut into small pieces by the help of a bistoury. 5 mg/ml of Collagenase type II enzyme was added into 10 mL of PBS and incubated at 37° C. for 3 hours. After incubation, the cells were centrifuged at 400×g. The cells and tissues were seeded into cell culture plates with antibiotic-containing media and adhesion of the cells was ensured for 3-4 days.

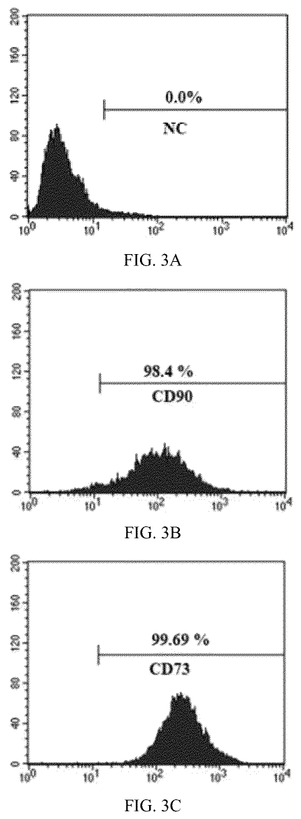

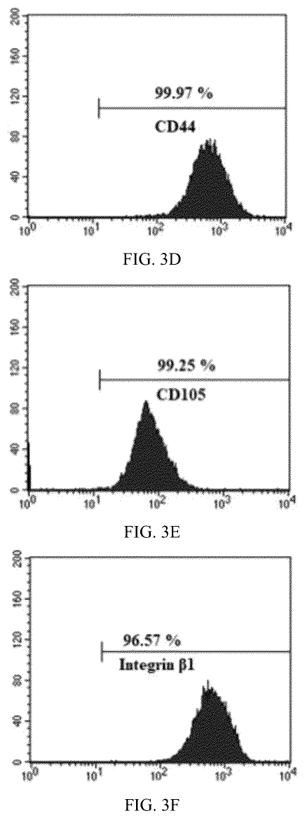

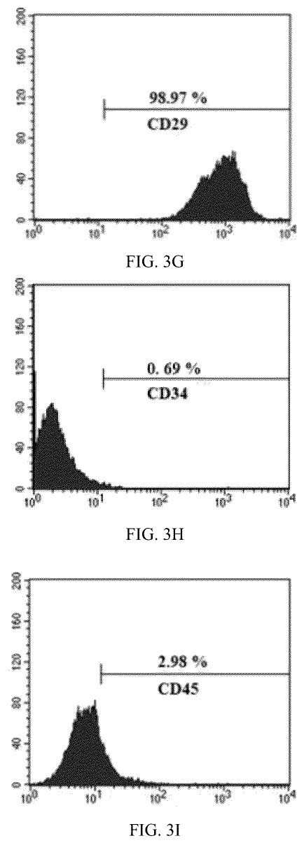

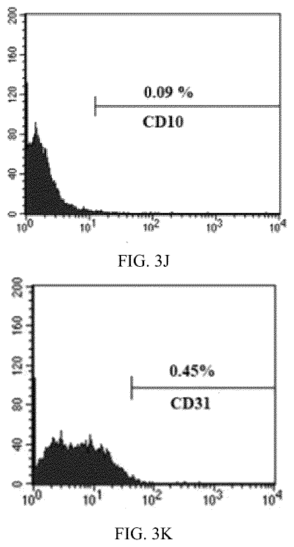

For characterization of the obtained stem cells, the cells were removed from the plates and divided into tubes. The cells were incubated for 30 minutes with CD90, CD73, CD44, CD105, integrin beta 1 (integrin (31), CD29, CD34, CD45, CD14 and CD31 surface markers and read in flow cytometry. (FIGS. 3A-3K)

### Developing the Treatment

### Determining the Cells Differentiating into Bone

In order to examine the differentiation of the product of the invention into bone cells, the ratio of the cells differentiating into bone cells was observed by Alizarin Red.

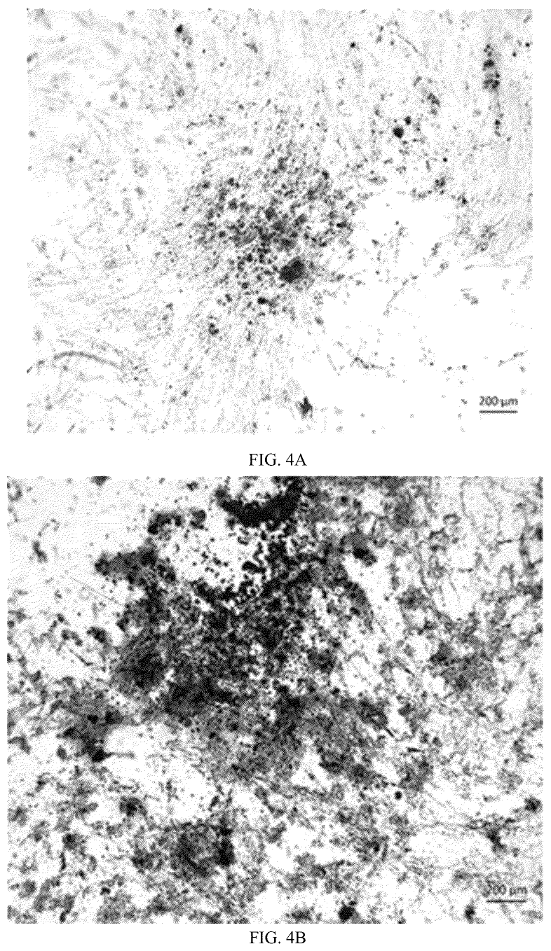

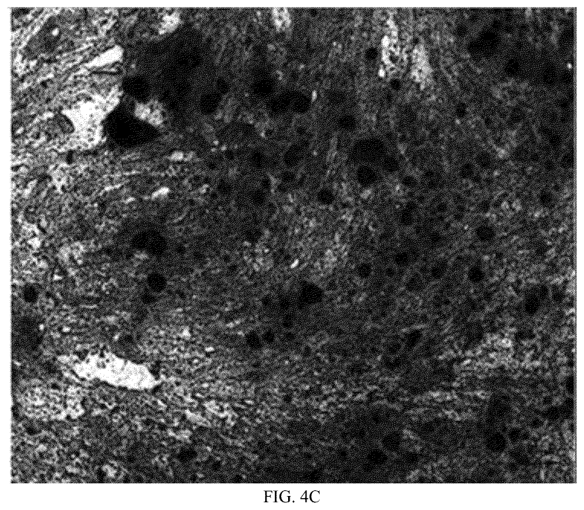

The hASCs (Adipose-derived stem cell) cell line was seeded in 48-well culture plates at 10,000 cells/well. While only differentiation media was given to the control cells, plant exosome was given to the other cells at concentrations of 100 μg/ml and 200 μg/ml together with the differentiation media. This procedure was repeated by changing the media of all cells every 3 days. At the end of 15 days, the wells were first washed with PBS. Then the cells were fixed to the surface by incubating in 4% Paraformaldehyde for 20 minutes. In order to view the cells that have differentiated into bone, 50 μl of 2% Alizarin Red was added to the wells and allowed to sit at room temperature for 15 minutes. After removing the stain, they were washed twice with distilled water. The cells were examined under a light microscope (FIGS. 4A-4C).

### Determining the Cells Differentiating into Cartilage

In order to examine the differentiation of the product of the invention into cartilage cells, the ratio of the cells differentiating into cartilage cells was observed by Alcian Blue.

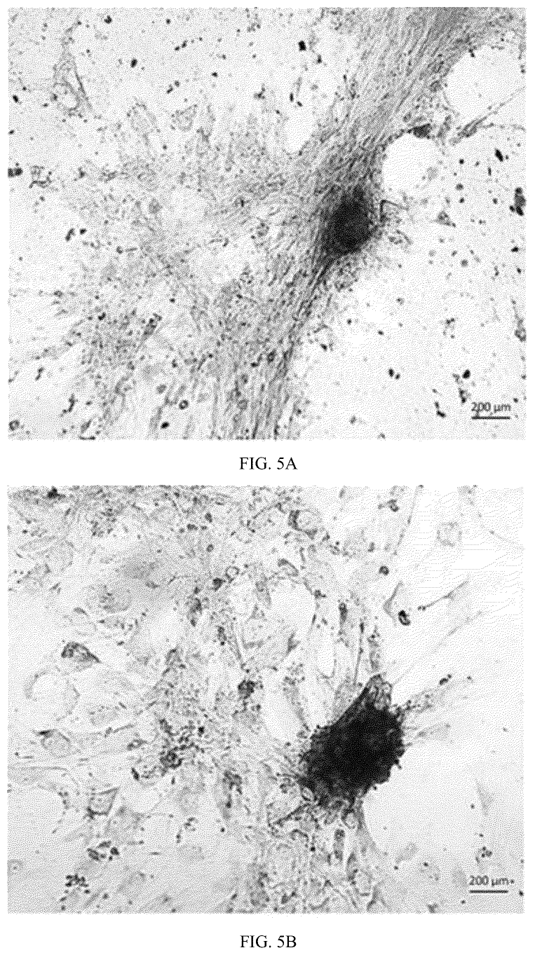

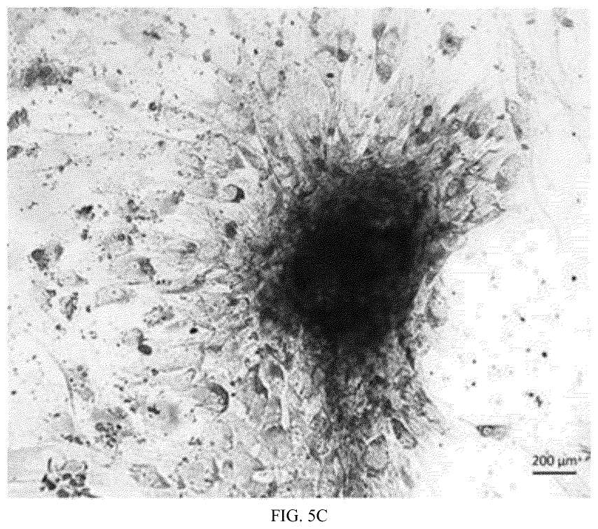

The hASCs (Adipose-derived stem cell) cell line was seeded in 48-well culture plates at 10,000 cells/well. While only differentiation media was given to the control cells, plant exosome was given to the other cells at concentrations of 100 μg/ml and 200 μg/ml together with the differentiation media. This procedure was repeated by changing the media of all cells every 3 days. At the end of 15 days, the wells were first washed with PBS. Then the cells were fixed to the surface by incubating in 4% Paraformaldehyde for 20 minutes. In order to view the cells that have differentiated into cartilage, 100 μl of Alcian Blue was added to the wells and allowed to sit at room temperature for 15 minutes. After removing the stain, they were washed twice with distilled water. The cells were examined under a light microscope. (FIGS. 5A-5C).

### Gene Expression Analysis of the Differentiated Cells

In order to examine the amounts of differentiation of the product of the invention into bone and cartilage, RNA was isolated from the differentiated cells and the differences in intracellular gene expression were examined.

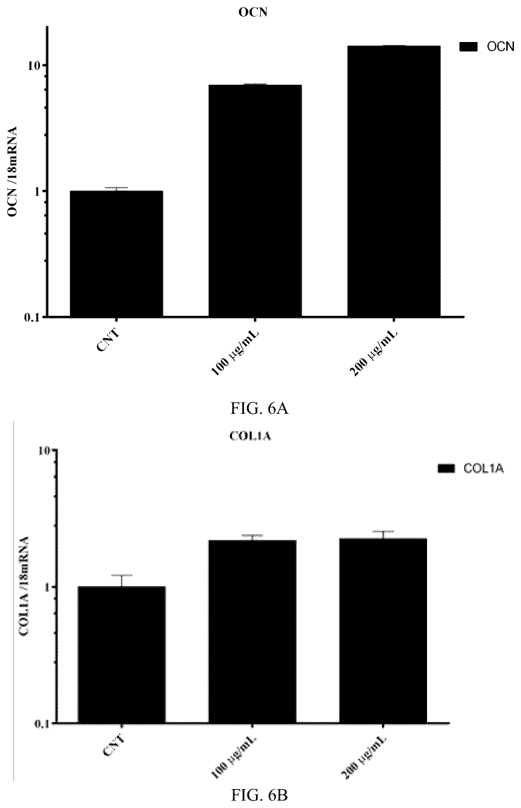

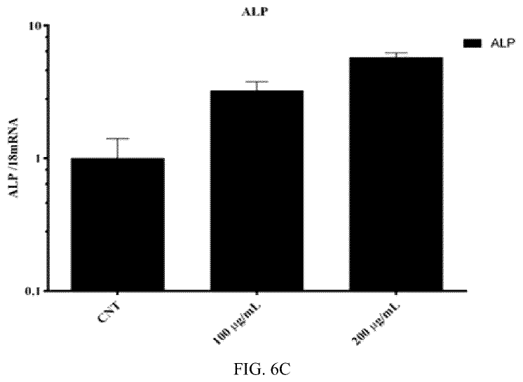

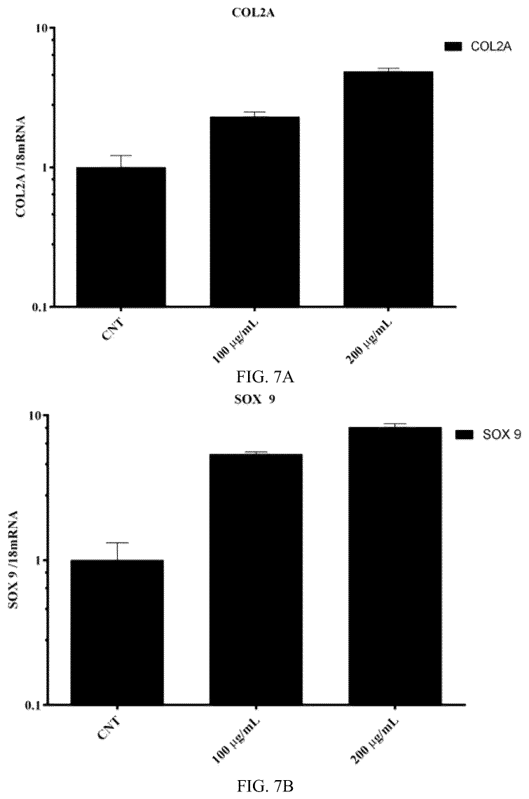

In the experimental study conducted to this end, human-derived adipose cells were seeded in 6-well culture plates at 100,000 cells per well. While only differentiation media was given to the control cells, plant exosome was given to the other cells at concentrations of 100 μg/ml and 200 μg/ml together with the differentiation media. This procedure was repeated by changing the media of all cells every 3 days. At the end of 15 days of differentiation, the cells were collected and RNAs were isolated using the Norgen RNA isolation kit. The isolated RNAs were converted to cDNA with the Bio-rad cDNA synthesis kit. For real-time Polymer Chain Reaction, 1.25 μl of cDNA, 2.9 μl of PCR water, 0.3 μl of forward primer, 0.3 μl of reverse primer and 5 μl of SYBR green were placed in each well. In order to examine the difference in expression of cells differentiating into bone, Osteocalcin, Collagen 1 and ALP genes (FIGS. 6A-6C) were observed, while for the cells that differentiate into cartilage, Collagen 2-A and Sox 9 genes (FIGS. 7A-7B) were observed. The 18s gene was used as the reference gene for all cells. The real-time PCR protocol was performed at the times and temperatures given in the table below.

| | | |
|---|---|---|
| Rpm | Temperature | Time |

| | | | | |
|---|---|---|---|---|
| 1 | 55° | C. | 15 | minutes |
| 2 | 95° | C. | 5 | minutes |
| 3 | 95° | C. | 60 | seconds |
| (39 repetitions) | 58° | C. | 60 | seconds |
| | 72° | C. | 60 | seconds |
| 4 | 72° | C. | 10 | minutes |
| 5 | 50-80° | C. | | |

| | |
|---|---|
| (80 repetitions) | 0.5° C. increase |
| | in 12 seconds |

| | | | |
|---|---|---|---|
| 6 | 4° | C. | ∞ |

In accordance with these results, it is seen that the plant exosome does not kill adipose cells, and it both accelerates the differentiation of stem cells into bone and cartilage and increases the yield thereof. Thanks to the invention, the bone and cartilage formation time is reduced to half, and at the same time, the formed bone and cartilage are enabled to be much more effective and stronger. In addition, a low cost and non-toxic product is provided. Apart from its therapeutic use, it is used as an osteogenic and chondrogenic differentiation medium supplement in in vitro experiments, and helps to achieve more effective results and shorten the experimental period.

What is claimed is:

1. A plant-derived exosome for a use in a differentiation of human-derived adipose cells (hASCs), tooth root stem cells, bone marrow stem cells, cartilage stem cells, endometrium stem cells, cord blood stem cells, and induced pluripotent stem cells (IPS cells) into bone cells and cartilage cells, wherein the plant-derived exosome is given to the hASCs, the tooth root stem cells, the bone marrow stem cells, the cartilage stem cells, the endometrium stem cells, the cord blood stem cells, and the IPS cells in addition to differentiation media to accelerate the differentiation into the cartilage cells and the bone cells and strengthen a formed bone and a formed cartilage.

2. The plant-derived exosome according to claim 1, wherein the plant-derived exosome is given to the hASCs seeded in a well culture plate at concentrations of 100 μg/ml and 20011 μg/ml in addition to the differentiation media to ensure the differentiation of the hASCs into the cartilage cells and the bone cells.

3. The plant-derived exosome according to claim 1, wherein the plant-derived exosome is used to accelerate a recovery after an osteoarthritis, an osteoporosis, a bone fracture, a cartilage destruction, and bone and cartilage surgeries up to 3-4 weeks, by accelerating the differentiation of the hASCs into a bone tissue and/or a cartilage tissue.

4. A method of obtaining the plant-derived exosome according to claim 1, comprising the following process steps:
grinding a plant-derived sample in a 1% phosphate buffered saline (PBS to obtain a ground plant-derived sample,
filtering the ground plant-derived sample to obtain a filtrate,
centrifuging the filtrate to obtain a centrifuged filtrate,
isolating the plant-derived exosome from the centrifuged filtrate by using an exosome isolation kit or an aqueous two-phase system (ATPS) method,
dissolving the plant-derived exosome in a 0.9% isotonic serum.

5. The method of obtaining the plant-derived exosome according to claim 4, wherein a wheatgrass collected after a growth of a seed of the wheatgrass for a period of 1.5 weeks is used as the plant-derived sample source for obtaining the plant-derived exosome.

6. A product comprising the plant-derived exosome according to claim 1, wherein the product is in a form of a serum, a syrup, a tablet, a drug, a gel, or a cream.

7. A method of obtaining the hASCs according to claim 1, comprising the process steps of
cutting an adipose tissue surgically removed from a human into small pieces by a bistoury,
adding 5 mg/ml of a Collagenase type II enzyme into 10 mL of a PBS to obtain a resulting solution and incubating the small pieces of the adipose tissue in the resulting solution at 37° C. for 3 hours to obtain incubated cells,
centrifuging the incubated cells at 400×g after the incubating to obtain centrifuged cells,
seeding the centrifuged cells into cell culture plates with antibiotic-containing media and ensuring an adhesion of the centrifuged cells for 3-4 days.

8. The plant-derived exosome according to claim 2, wherein the plant-derived exosome is used to accelerate a recovery after an osteoarthritis, an osteoporosis, a bone fracture, a cartilage destruction, and bone and cartilage surgeries up to 3-4 weeks, by accelerating the differentiation of the hASCs into a bone tissue and/or a cartilage tissue.

9. The method of obtaining the plant-derived exosome according to claim 4, wherein the plant-derived exosome is given to the hASCs seeded in a well culture plate at concentrations of 100 μg/ml and 200 μg/ml in addition to the differentiation media to ensure the differentiation of the hASCs into the cartilage cells and the bone cells.

10. The method of obtaining the plant-derived exosome according to claim 4, wherein the plant-derived exosome is used to accelerate a recovery after an osteoarthritis, an osteoporosis, a bone fracture, a cartilage destruction, and bone and cartilage surgeries up to 3-4 weeks, by accelerating the differentiation of the hASCs into a bone tissue and/or a cartilage tissue.

11. The product according to claim 6, wherein the plant-derived exosome is given to the hASCs seeded in a well culture plate at concentrations of 100 μg/ml and 200 μg/ml in addition to the differentiation media to ensure the differentiation of the hASCs into the cartilage cells and the bone cells.

12. The product according to claim 6, wherein the plant-derived exosome is used to accelerate a recovery after an osteoarthritis, an osteoporosis, a bone fracture, a cartilage destruction, and bone and cartilage surgeries up to 3-4 weeks, by accelerating the differentiation of the hASCs into a bone tissue and/or a cartilage tissue.

13. The product according to claim 6, wherein a method of obtaining the plant-derived exosome comprises the following process steps:
grinding a plant-derived sample in a 1% phosphate buffered saline (PBS) to obtain a ground plant-derived sample,
filtering the ground plant-derived sample to obtain a filtrate,
centrifuging the filtrate to obtain a centrifuged filtrate,
isolating the plant-derived exosome from the centrifuged filtrate by using an exosome isolation kit or an aqueous two-phase system (ATPS) method,
dissolving the plant-derived exosome in a 0.9% isotonic serum.

14. The product according to claim 13, wherein a wheatgrass collected after a growth of a seed of the wheatgrass for a period of 1.5 weeks is used as the plant-derived sample for obtaining the plant-derived exosome.
